(19) United States
(12) Patent Application Publication
(10) Pub. No.: US 2013/0289247 A1
(43) Pub. Date: Oct. 31, 2013

(54) SINGLE UNIT ION EXCHANGE CHROMATOGRAPHY ANTIBODY PURIFICATION

(71) Applicants: Diderik Reinder Kremer, Groningen (NL); Marijke Yvonne Dorst, Groningen (NL)

(72) Inventors: Diderik Reinder Kremer, Groningen (NL); Marijke Yvonne Dorst, Groningen (NL)

(73) Assignee: DSM IP ASSETS B.V., Heerlen (NL)

(21) Appl. No.: 13/881,201

(22) PCT Filed: Oct. 13, 2011
(86) PCT No.: PCT/EP2011/067882

§ 371 Date: Jul. 15, 2013

(30) Foreign Application Priority Data
Nov. 1, 2010 (EP) 10189563.9

(51) Int. Cl. C07K 1/18

(52) U.S. Cl. CPC C07K 1/18
USPC 530/387.1; 210/198.2

(57) ABSTRACT

The present invention relates to a method for the purification of antibodies from a protein mixture produced in a bioreactor, at least comprising the steps of intermediate purification and polishing, wherein the intermediate and polishing step comprises in either order in-line anion exchange chromatography (AEX) chromatography and cation exchange chromatography (CEX) chromatography steps in flow-through mode. The present invention further relates to a single operational unit comprising both an anion exchange chromatography part and a cation exchange chromatography part in either order, which are serially connected, wherein the unit comprises an inlet at the upstream end of the first ion exchange chromatography part and an outlet at the downstream end of the second ion exchange chromatography part and wherein the unit also comprises an inlet between the first ion exchange chromatography part and the second ion exchange chromatography part.

The present invention relates to a method for single unit purification of antibodies and to equipment which can be used in this method.

The purification of monoclonal antibodies, produced by cell culture, for use in pharmaceutical applications is a process involving a large number of steps. The antibodies are essentially to be freed from all potentially harmful contaminants such as proteins and DNA originating from the cells producing the antibodies, medium components such as insulin, PEG ethers and antifoam as well as any potentially present infectious agents such as viruses and prions.

Typical processes for purification of antibodies from a culture of cells producing these proteins are described in BioPharm International Jun. 1, 2005, “Downstream Processing of Monoclonal Antibodies: from High Dilution to High Purity.”

As antibodies are produced by cells, such as hybridoma cells or transformed host cells (like Chinese Hamster Ovary (CHO) cells, mouse myeloma-derived NS0 cells, Baby Hamster Kidney cells and human retina-derived PER.C6® cells), the particulate cell material will have to be removed from the cell broth, preferably early in the purification process. This part of the process is indicated here as “clarification”. Subsequently or as part of the clarification step the antibodies are purified roughly to at least about 80%, usually with a binding plus eluting chromatography step (in the case of IgG often using immobilized Protein A). This step, indicated here as “capturing” not only results in a first considerable purification of the antibody, but may also result in a considerable reduction of the volume, hence concentration of the product. Alternative methods for capturing are for example Expanded Bed Adsorption (EBA), 2-phase liquid separation (using e.g. polyethyleneglycol) or fractionated precipitation with lyotropic salt (such as ammonium sulfate).

Subsequent to clarification and capturing, the antibodies are further purified. Generally, at least 2 chromatographic steps are required after capturing to sufficiently remove the residual impurities. The chromatographic step following capturing is often called intermediate purification step and the final chromatographic step generally is called the polishing step. Each of these steps is generally performed as single unit operation in batch mode and at least one of these steps is carried out in the binding plus eluting mode. In addition, each chromatographic step requires specific loading conditions with respect to e.g. pH, conductivity etc. Therefore, extra handling has to be performed prior to each chromatography step in order to adjust the load to the required conditions. All of this mentioned makes the process elaborate and time consuming. The impurities generally substantially removed during these steps are process derived contaminants, such as host cell proteins, host cell nucleic acids, culture medium components (if present), protein A (if present), endotoxin (if present), and micro-organisms (if present). Many methods for such purification of antibodies have been described in recent patent publications.

WO 2010/062244. The invention relates to an aqueous two phase extraction augmented precipitation process for isolation and purification of proteins like monoclonal antibodies. For subsequent further purification of antibodies are described two alternatives: (1) cation exchange chromatography in bind and elute mode, followed by anion exchange in flow through mode, or (2) first multimodal chromatography in flow through mode, followed by anion exchange in flow-through mode.

WO 2010/048183. The invention relates to the removal of HCP from antibodies by consecutive ion exchange at acid pH and HIC chromatography.

WO 2009/138484. At first reading the invention is not clear from the description and claims. It relates to the purification of antibodies from a mixture by capture of the antibodies on a Protein A (derivative) column and subsequent release of the antibodies from this column. This latter antibody-containing material can be further purified e.g. by consecutive anion chromatography and cation chromatography.

EP 2 027 921 The invention relates to media for membrane ion exchange chromatography based on polymeric primary amines, and the use thereof in purification of e.g. antibodies.

WO 2005/044856 relates to the removal of high-molecular weight aggregates from an antibody preparation, using a hydroxyapatite resin optionally in combination with anion exchange chromatography.

WO2008/145351 describes subsequent anion exchange chromatography and cation exchange chromatography both in flow through mode.

Disadvantages of the methods described above are long operation time, high variable costs (for example due to the necessity of large column capacity, which is inherently required for a binding plus eluting step, and hence large amounts of costly resins needed) and high fixed cost (due to labor costs).

According to one embodiment of the present invention, very efficient removal of residual impurities from cell culture-produced antibodies can be achieved by using serial, in-line anion exchange chromatography (AEX) and cation exchange chromatography (CEX) both in the flow-through mode and preferably operating as one single unit operation. Therefore, in-line mixing of a suitable buffer after the AEX and before the CEX chromatographic step is used to adjust the right conditions with respect to pH and conductivity for the CEX chromatography.

According to a further embodiment of the present invention, very efficient removal of residual impurities from cell culture-produced antibodies can be achieved by using serial, in-line cation exchange chromatography (CEX) and anion exchange chromatography (AEX) both in the flow-through mode and preferably operating as one single unit operation. Therefore in-line mixing of a suitable buffer after the CEX and before the AEX chromatographic step is used to adjust the right conditions with respect to pH and conductivity for the AEX chromatography.

Advantages of this novel method are considerable reduction of the operation time and labor and hence lower operational costs. In addition, smaller (and thus less costly) chromatographic units are required, since all units operate in flow-through mode which requires only sufficient binding capacity for the impurities and not for the product.

Therefore, the present invention can be defined as a method for the purification of antibodies from a cell broth produced in a bioreactor, at least comprising the steps of intermediate purification and polishing, wherein the novel purification step comprises combined serial in-line AEX and CEX chromatography. This can be carried out in either of two alternative ways (1) as anion exchange (AEX) chromatography yielding as a flow-through fraction a separation mixture, serial in-line followed by cation exchange (CEX) chromatography yielding as a flow-through fraction a purified antibody preparation, or (2) as cation exchange (CEX) chromatography yielding as a flow-through fraction a separation mixture, serial in-line followed by anion exchange (AEX) chromatography yielding as a flow-through fraction a purified antibody preparation and wherein the purified antibody preparation resulting from either of these alternative ways is subjected to at least one further purification step.

WO2008/145351 describes also subsequent anion exchange chromatography and cation exchange chromatography both in flow through mode, in any order. However, its disclosure differs from the present invention in that the conditioning of the separation mixture from the first separation step to prepare it for the second separation step is carried out off-line. It is surprising that according to the present invention the integration of both chromatographic processes could be done so well that both ion exchange process flows could be tuned mutually and at the same time the adjustment of the buffer conditions for the second chromatography step could be carried out so accurate that a complete clearance of aggregates was achieved.

In the context of the present invention, the “separation mixture” is the solution resulting from the first ion exchange step according to the invention, and the “purified antibody preparation” is the solution resulting from the second ion exchange step according to the invention. It is intended to adhere to this terminology throughout the present application.

Prior to the first ion exchange chromatography step, the cell broth produced in the bioreactor generally will be clarified (i.e. freed from all cellular material, such as whole cells and cell debris).

Also, prior to the first ion exchange chromatography step, a conditioning solution may be added to the cell broth or the antibody containing solution in order to ensure optimum conditions in terms of pH and conductivity for this first ion exchange step.

In a particular embodiment the method according to the invention involves that the combined chromatography with AEX and CEX is performed as a single unit operation.

With “flow-through fraction” is meant here at least part of the loaded antibody-containing fraction which leaves the chromatographic column at substantially the same velocity as the elution fluid. This fraction is substantially not retained on the column during elution. Hence the conditions are chosen such that not the antibodies but the impurities are bound to the anion exchange material and to the cation exchange material.

Separation of proteins using subsequent chromatography of the protein mixture with anion exchange and cation exchange interaction chromatography has been disclosed, e.g. in WO 2009/138484 as discussed above. Therein the two ion exchange steps were carried out as two separate steps.

It has been found that for large scale production purposes the method according to the present invention (with flow-through mode) provides a much faster separation than the prior disclosed method with binding and elution of the desired antibodies.

Advantageously, the separation mixture containing the antibody is supplemented with an adequate amount of solution in order to adjust the pH and conductivity for optimum performance in the second ion exchange chromatography step according to the present invention.

Surprisingly it was found that very good separation results can be achieved with in-line conditioning of the fluid before entering into the second ion exchange step.

When AEX chromatography is the first step, generally, the AEX is carried out at slightly alkaline pH and at low conductivity. We found that the performance of the CEX in flow-through mode was best at slightly acidic conditions at low conductivity. So, therefore the flow-through product from AEX chromatography is supplemented in-line with an acidic solution decreasing the pH to the desired value and adjusting or maintaining the optimum conductivity before it will be subjected to the CEX chromatography. Any solution or buffer that will result in an adequate pH decrease and conductivity adjustment may be used to this end. Preferably, the pH is corrected to a value of at least about 3.5, more preferably to a value of at least about 4, more preferably to a value of at least about 5. Preferably, the pH is corrected to a maximum pH value of about 7. The conductivity preferably is maintained at or corrected to at least about 2 mS, and at maximum is about 10 mS. Preferably, the solution contains an acidic component that requires a small amount to be supplemented resulting in minimum dilution of the product. The acidic component may be chosen from compounds such as citric acid (or its mono or di-basic sodium or potassium salts), phosphoric acid (or its mono or di-basic sodium or potassium salts), acetic acid, hydrochloric acid, sulfuric acid.

Preferably, supplementing the separation mixture in this case with an adequate amount of pH and conductivity adjusting solution is part of the single unit operation e.g. by in-line mixing of mentioned acidic solution in the process stream (e.g. in a mixing chamber) prior to the CEX chromatography step.

With “an adequate amount of an acidic solution” is meant here sufficient mentioned solution to cause adsorption of the majority of relevant impurities to the CEX material, but an amount that is low enough not to cause binding of the product. For each purification process the optimum amount and preferred type of acidic components have to be established.

Alternatively, the sequence of AEX and CEX can be changed. In this case, the process starts with a CEX chromatography unit and the antibody-containing solution from this CEX chromatography should be pre-conditioned at such pH and conductivity that optimum purification takes place in this CEX unit in flow-through mode. Generally, this will be at slightly acidic pH and at low conductivity. The subsequent AEX step must be carried out at optimum purification conditions for that specific step. Preferably, the pH is corrected to a value of at maximum about 9, more preferably to a value of at maximum about 9.5. Preferably, the pH is corrected to at least a pH value of about 7. The conductivity preferably is maintained at or corrected to at least about 2 mS, and at maximum is about 10 mS. Generally, this will be at slightly alkaline pH and a low conductivity. To this end the antibody-containing solution after CEX and prior to AEX chromatography is supplemented in-line with an adequate amount of solution in order to adjust the pH and conductivity for optimum AEX performance. So, in practice the flow-through product from CEX chromatography is supplemented in-line with an alkaline solution to increase the pH of the separation mixture to the desired value and adjusting or maintaining the optimum conductivity for operation of the AEX chromatography unit. Any solution or buffer that will result in an adequate pH decrease and conductivity adjustment may be used to this end. Preferably, the solution contains an alkaline component that requires a small amount to be supplemented resulting in minimum dilution of the product. Some examples of such alkaline components are sodium or potassium hydroxide, (or its mono or di basic sodium or potassium salts), tris(hydroxymethyl)aminomethane, but any other alkaline component known in the art may be used to this end.

AEX chromatography according to the invention may take place in an AEX unit which may be embodied by a classical packed bed column containing a resin, a column containing monolith material, a radial column containing suitable chromatographic medium, an adsorption membrane unit, or any other anion exchange chromatography device known in the art with the appropriate medium and ligands to function as an anion exchanger. In the AEX column the chromatographic material may be present as particulate support material to which strong or weak cationic ligands are attached. The membrane-type anion exchanger consists of a support material in the form of one or more sheets to which strong or weak cationic ligands are attached. The support material may be composed of organic material or inorganic material or a mixture of organic and inorganic material. Suitable organic materials are agarose based media and methacrylate. Suitable inorganic materials are silica, ceramics and metals. A membrane-form anion exchanger may be composed of hydrophilic polyethersulfone containing AEX ligands. Suitable strong AEX ligands are based e.g. on quaternary amine groups. Suitable weak AEX ligands are based on e.g. primary, secondary or tertiary amine groups or any other suitable ligand known in the art.

CEX chromatography according to the invention may take place in an CEX unit which may be embodied by a classical column containing a resin, a column based on monolith material, a radial column containing suitable chromatographic medium, an adsorption membrane unit, or any other cation exchange chromatography device known in the art with the appropriate ligands to function as a cation exchange material. In the CEX column the chromatographic material may be present as particulate support material to which CEX ligands are attached. The membrane-like chromatographic device consists of a support material in the form of one or more sheets to which CEX ligands are attached. The support material may be composed of organic material or inorganic material or a mixture of organic and inorganic material. Suitable organic support materials are composed of e.g. hydrophilic carbohydrates (such as cross-linked agarose, cellulose or dextran) or synthetic copolymer materials (such as poly(alkylaspartamide), copolymers of 2-hydroxyethyl methacrylate and ethylene dimethacrylate, or acylated polyamine). Suitable inorganic support materials are e.g. silica, ceramics and metals. A membrane-form CEX may be composed of hydrophilic polyethersulfone containing CEX ligands. Suitable examples of CEX ligands are sulfonic acid, carboxylic acid, phosphinic acid or any other ligand known in the art to function as a strong or weak cation exchanger.

Antibodies which can be purified according to the method of the present invention are antibodies which have an isoelectric pH of 6.0 or higher, preferably 7.0 or higher, more preferably 7.5 or higher. These antibodies can be immunoglobulins of either the G, the A, or the M class. The antibodies can be human, or non-human (such as rodent) or chimeric (e.g. “humanized”) antibodies, or can be subunits of the abovementioned immunoglobulins, or can be hybrid proteins consisting of an immunoglobulin part and a part derived from or identical to another (non-immunoglobin) protein.

Surprisingly, the antibody material resulting from the combined AEX and CEX chromatography generally will have a very high purity (referring to protein content) of at least 98%, preferably at least 99%, more preferably at least 99.9%, even more preferably at least 99.99%.

The anion exchange chromatography step according to the present invention preferably is carried out at neutral or slightly alkaline pH. It will remove the negatively charged impurities like DNA, host cell proteins, protein A (if present), viruses (if present), proteinacous medium components such as insulin and insulin like growth factor (if present).

During the cation exchange chromatography step the major remaining large molecular impurities (mainly product aggregates) will be removed, using the property that, applying the right the conditions of pH and, conductivity, they bind to the chromatographic device while the product flows through.

Subsequently, the (highly) purified antibody preparation will, generally, have to be treated by ultrafiltration and diafiltration, in order to remove all residual low molecular weight impurities, to replace the buffer by the final formulation buffer and to adjust the desired final product concentration.

Furthermore, the purified antibody preparation will, generally, have to be treated also to assure complete removal of potentially present infectious agents, such as viruses and/or prions.

The present invention also relates to a single operational unit comprising both an anion exchange chromatography part (AEX) and a cation exchange chromatography part (CEX), which are serially connected. This single operational unit further comprises an inlet at the upstream end of the first ion exchange chromatography part and an outlet at the downstream end of the second ion exchange chromatography part. This single operational unit also comprises a connection between the first ion exchange chromatography part and the second ion exchange chromatography part further comprising an inlet for supply of a conditioning solution to the separation mixture.

Accordingly, in one embodiment thereof the invention relates to a single operational unit which can be used in a method according to the invention comprising both an anion exchange chromatography part and a cation exchange chromatography part, which are in this order serially connected, wherein the outlet of the anion exchange chromatography part is connected to the inlet of the cation exchange chromatography part, wherein the unit comprises an inlet at the upstream end of the anion exchange chromatography part and an outlet at the downstream end of the cation exchange chromatography part and wherein the unit also comprises an inlet between the anion exchange chromatography part and the cation exchange chromatography part for supply of an acidic conditioning solution to the separation mixture.

In a further embodiment the invention relates to a single operational unit which can be used in a method according to the invention comprising both an cation exchange chromatography part and a anion exchange chromatography part, which are in this order serially connected, wherein the outlet of the cation exchange chromatography part is connected to the inlet of the anion exchange chromatography part, wherein the unit comprises an inlet at the upstream end of the cation exchange chromatography part and an outlet at the downstream end of the anion exchange chromatography part and wherein the unit also comprises an inlet between the cation exchange chromatography part and the anion exchange chromatography part for supply of an alkaline conditioning solution to the separation mixture.

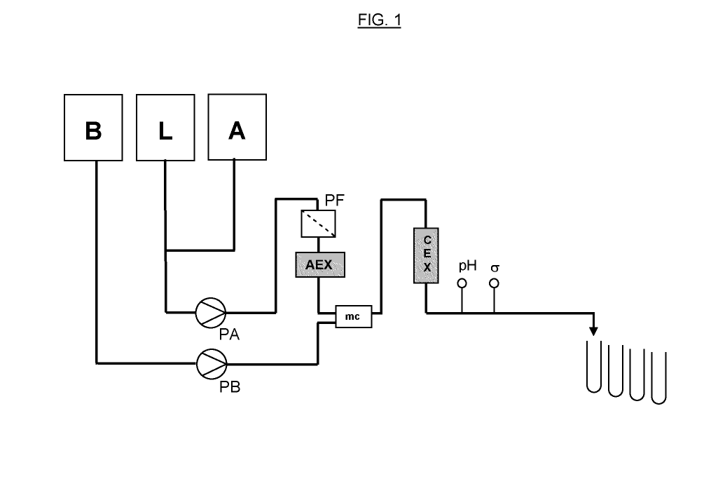

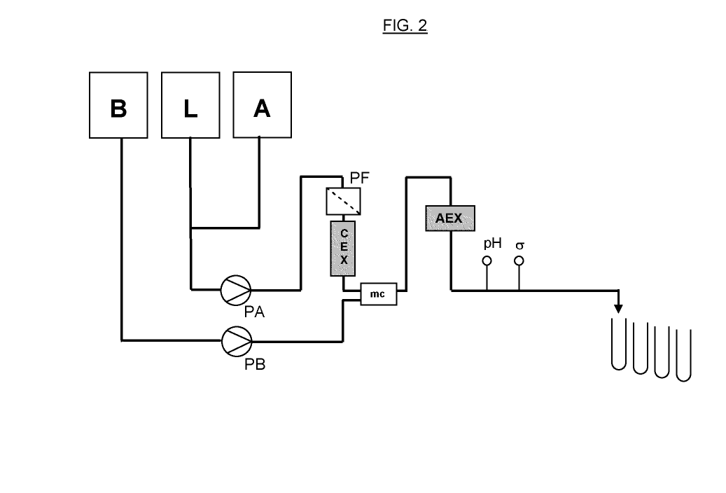

The liquid flow during the process according to the present invention can be established by any dual pump chromatographic system commercially available, e.g. an ÅKTA explorer (GE), a BIOPROCESS (GE) any dual pump HPLC system or any tailor made device complying with the diagram of FIG. 1 or 2. Most of these chromatographic devices are designed to operate a single chromatographic unit (i.e. column or membrane). With a simple adaptation, an extra connection can be made to place the first ion exchange unit after pump A and before the mixing chamber.

FIGS. 1 and 2 display the basic configurations. Serial inline connection of two chromatographic devices plus an optional pre-filter in the position as shown in FIGS. 1 and 2, may lead to undesirable pressure buildup. Therefore, under some conditions extra technical adaptations (e.g. an extra pump after the AEX unit and a pressure reducing device before the AEX unit) may have to be included into this diagram.

### DESCRIPTION OF THE FIGURES

FIG. 1. A single operational unit comprising both an anion exchange chromatography part and a cation exchange chromatography part. Buffer A is a conditioning and washing buffer suitable for optimum operation of the AEX step. Buffer B contains an acidic solution and is mixed in a ratio to the load/buffer A required to obtain optimum conditions for operation of the CEX step. The mixing ratio can be executed using a fixed volumetric mixing flow or can be automatically controlled by a feed back loop, based on e.g. the pH output. MC is an optional mixing chamber, which may contain any type of static mixer.
L=Load
PA=Pump A
PB=Pump B
AEX=anion exchange unit
CEX=cation exchange unit
pH=pH sensor
σ=conductivity sensor
PF=optional pre-filter

FIG. 2. A single operational unit comprising both an cation exchange chromatography part and a anion exchange chromatography part. Buffer A is a conditioning and washing buffer suitable for optimum operation of the CEX step. Buffer B contains an alkaline solution and is mixed in a ratio to the load/buffer A required to obtain optimum conditions for operation of the AEX step. The mixing ratio can be executed using a fixed volumetric mixing flow or can be automatically controlled by a feed back loop, based on e.g. the pH output. MC is an optional mixing chamber, which may contain any type of static mixer.
L=Load
PA=Pump A
PB=Pump B
AEX=anion exchange unit
CEX=cation exchange unit
pH=pH sensor
σ=conductivity sensor
PF=optional pre-filter

### EXAMPLES

### Materials and Methods

All experiments were carried out using an IgG1 produced by a mammalian cell line.

The cultivation was carried out applying XD® culture, (see Genetic Engineering & Biotechnology News, Apr. 1 2010 Vol. 30, No. 7) using a chemically defined medium and afterwards the harvest was diluted and cells were removed by a three step depth filtration filter train ZetaPlus 10M02P, ZetaPlus 60ZA05 and SterAssure PSA020 all from Cuno (3M).

This clarified harvest contained approximately 4.0 g/L IgG and was stored in aliquots at −20 ° C. It was thawed and equilibrated to room temperature prior to protein A purification.

First an initial purification by standard Protein A chromatography was carried out using MabSelect (GE) with standard procedures (loading clarified harvest, first wash with 20 mM Tris+150 mM NaCl, second wash with 100 mM sodium acetate buffer at pH 5.5 and elution with 100 mM acetate buffer pH 3.0).

After MabSelect elution, the eluted peak was collected and maintained for 1 hour at pH 3.5. After that, the sample was neutralized to pH 7.4 using 2M Tris pH 9.0 and was filtered over 0.22 μm.

With material thus obtained, 3 series of experiments were carried out: 1. to establish HCP removal performance for AEX chromatography in flow-through mode (Experiment 1). 2. to establish optimum conditions using CEX chromatography in flow-through mode (Experiment 2). 3. combining both optimized AEX and CEX conditions in one single unit operation experiment (Example 1).

HCP was measured by ELIZA with polyclonal anti-PerC6 HCP. Monomeric IgG and aggregate concentrations were determined by size exclusion chromatography (HP-SEC) according to standard procedures.

### Experiment 1

### Establishing Conditions for Good Performance of Anion Exchange Chromatography in Flow-Through Mode

AEX chromatography in flow-through mode was carried out using mentioned pre-purified IgG in acetate Tris buffer. The following AEX media were tested: Mustang Q coin (0.35 ml) (PaII), Sartobind Q capsule (1 ml) and ChromaSorb capsule (0.08 ml) (Millipore) (all membrane adsorbers).

All AEX media were run in flow-through using an ÅKTA explorer at 40 bed volumes/hr. The samples were diluted with demineralized water up to a final conductivity of 5 mS. Conditioning and washing buffer was 100 mM acetate Tris pH 7.4 The amount of product loaded on each AEX medium was 1.5 g IgG/mL membrane bed volume.

HCP was measured before and after the chromatography steps. The starting material contained 3305 ng/mg IgG. The eluted material contained 39, 57 and 71 ng/mg IgG, respectively for the mentioned Mustang Q, Sartobind Q and Chromasorb membranes. These results clearly showed that all tested AEX chromatographic media, sufficiently removed HCP's under the applied conditions.

### Experiment 2

### Establishing Conditions for Aggregate Removal Using CEX Chromatography in Flow-Through Mode

For these experiments the pre-purified IgG was adjusted to pH 8.0 and diluted to a conductivity of 2 mS. A VL11 (Millipore) column filled with 16 cm bed length Poros 50HS (Applied Biosystems) was used on an ÅKTA explorer. The wash and equilibration buffer was 50 mM Tris HCl pH 7.4 at a flow of 5.35 ml/min. After the wash, the product was loaded at a similar flow rate. During the loading, with a second pump, a solution of 50 mM NaH₂PO₄ (buffer B) was mixed in-line before the column in order to adjust pH and conductivity before entering the column. Different fixed ratios of product flow and buffer B were tested, maintaining the overall flow over the column constant. Samples of the flow-through were taken at each ratio after that the UV signal was stabilized.

TABLE 1
Aggregate clearance with Poros 50HS using different volume ratios
of in-line mixing of 50 mM NaH₂PO₄ containing buffer B.

| % buffer B | pH | conductivity (mS) | Aggregates (%) | IgG (g/L) |
|---|---|---|---|---|
| 0% | 7.96 | 1.94 | 3.29 | 1.33 |
| 17.5% | 7.68 | 2.58 | 3.24 | 1.29 |
| 22.5% | 7.47 | 2.72 | 2.52 | 1.28 |
| 27.5% | 7.18 | 2.89 | 1.82 | 1.20 |
| 37.5% | 6.83 | 3.00 | 0.16 | 0.98 |
| 42.5% | 6.68 | 3.08 | 0.00 | 0.96 |

The analytical results on these samples (shown in Table 1) clearly show that when the pH is sufficiently decreased the flow-through does not contain aggregates anymore, while it still contains IgG.

### Example 1

### Purification of IgG at Optimized AEX and CEX Conditions in One Single Unit Operation

An AEX unit and a CEX unit were serially coupled in-line as depicted in the diagram of FIG. 1 using an ÅKTA explorer. For the AEX, a Sartobind Q capsule (1 mL) was used and for the CEX a VL11 (Millipore) column filled with 16 cm bed length Poros 50HS (Applied Biosystems) was used. For conditioning before product loading a 50 mM Tris HCl buffer pH 7.4, conductivity 4.0 mS was applied (buffer A). Simultaneously, buffer B was mixed in-line at a 27.5% volume ratio after the AEX membrane and before the CEX resin. Buffer B contained 50 mM NaH₂PO₄. The overall flow over the CEX unit was 5.35 mL/min.

For this experiment the pre-purified IgG was diluted with demineralized water to a conductivity of 2.98 mS. The loading of the pre-purified IgG was started by pumping the IgG at a similar flow as buffer A, while buffer A pumping was stopped. Buffer B was maintained at a flow of 27.5% volume ratio. An amount of 602.5 mL containing 1.96 g IgG was loaded. After completing the loading, the flow was switched back to buffer A, in order to recover all product from the system (the wash). After washing, the AEX-CEX unit was stripped by adding 2M NaCl via pump A and pump B was stopped. The strip was separately collected. During the whole run the flow over the CEX was 5.35 ml/min. The total time (including conditioning, washing and stripping) was less than 3 hours. Both the load and the flow-through were analyzed for IgG aggregate ratio, and HCP content and protein (product) content (A²⁸⁰). The HCP concentration was 2697 ng/mg IgG in the starting material and was ≦47 ng/mg IgG in the flow-through plus wash fraction. The amount of aggregates was 3.66% in the load (starting material) and was 0.00% in the flow-through plus wash, showing complete aggregate clearance. The strip contained 30.4% aggregates. The overall product recovery in the flow-through plus wash was not assessed, but in a previously carried out comparable run it was approximately 86% in the flow-through plus wash and 92% in the flow-through plus wash plus strip.

### Example 2

### Purification of IgG at Optimized CEX and AEX Conditions in One Single Unit Operation

A CEX unit and an AEX unit are serially coupled in-line as depicted in the diagram of FIG. 2 using an ÅKTA explorer. For the CEX a VL11 (Millipore) column filled with 16 cm bed length Poros 50HS (Applied Biosystems) is used and for the AEX unit a Sartobind Q capsule (1 mL) is used. For conditioning before product loading a 50 mM Tris-acetate buffer pH 6.6, conductivity 4.0 mS is applied (buffer A). Simultaneously, buffer B was mixed in-line at a 20% volume ratio after the AEX membrane and before the CEX resin. Buffer B contains 200 mM Tris pH 9.0. The overall flow over the AEX unit is 5.4 mL/hr.

For this experiment the pre-purified IgG pH is adjusted to 6.6 in stead of 7.4 and is subsequently diluted with demineralized water to a conductivity of 4 mS. The loading of this pH 6.6 adjusted pre-purified IgG is started by pumping the IgG at a similar flow as buffer A, while the buffer A flow is stopped. Buffer B is maintained a flow of 20% volume ratio. An amount of 600 ml containing 1.5 g IgG is loaded. After completing the loading, the flow is switched back to buffer A, in order to recover all product from the system (the wash). After washing, the CEX-AEX unit is stripped by adding 2M NaCl via pump A and pump B is stopped. The strip is separately collected. During the whole run the flow over the AEX is 5.4 ml/hr. The total time (including conditioning, washing and stripping) is less than 3 hours. Both the load and the flow-through are analyzed for IgG aggregate ratio, and HCP content and protein (product) content (A²⁸⁰).

### Abbreviations Used

A280 (Light) Absorption at 280 nm
AEX Anion Exchange
CEX Cation exchange
CHO cells Chinese Hamster Ovary cells
EBA Expanded Bed Adsorption
HCP Host Cell Protein
HPLC High Pressure Liquid Chromatography
IgG Immunoglobulin G
LoD Limit of Detection
TFF Tangential Flow Filtration
Tris tris(hydroxymethyl)methylamine

1-11. (canceled)

12. A method for the purification of antibodies from a cell broth produced in a bioreactor, at least comprising the steps of intermediate purification and polishing, wherein the intermediate purification step comprises combined serially connected in-line anion exchange (AEX) and cation exchange (CEX) chromatography steps in either order, both in flow-through and together operating as one single unit operation, wherein the first chromatography step yields as a flow-through fraction a separation mixture, which is directly subjected to the second chromatography step wherein the second step yields as a flow through fraction a purified antibody preparation, and wherein the purified antibody preparation is subjected to at least one further purification step.

13. A method according to claim 12, wherein the AEX chromatography step is carried out first, yielding as a flow-through fraction a separation mixture, serial in-line followed by a CEX chromatography step yielding as a flow-through fraction a purified antibody preparation and wherein the purified antibody preparation is subjected to at least one further purification step.

14. A method according to claim 12, wherein the CEX chromatography step is carried out first, yielding as a flow-through fraction a separation mixture, serial in-line followed by a AEX chromatography step yielding as a flow-through fraction a purified antibody preparation and wherein the purified antibody preparation is subjected to at least one further purification step.

15. Method according to claim 12, wherein the separation mixture prior to the second ion exchange step is supplemented with an adequate amount of solution in order to adjust the pH and conductivity for optimum performance in the second ion exchange chromatography step.

16. Method according to claim 13, wherein the separation mixture prior to CEX chromatography is supplemented with an adequate amount of an acidic solution.

17. Method according to claim 16, wherein the separation mixture prior to CEX chromatography is supplemented with an adequate amount of a solution containing citric acid (or its monobasic or dibasic sodium or potassium salts), phosphoric acid (or its monobasic or dibasic sodium or potassium salts), acetic acid, hydrochloric acid or sulfuric acid.

18. Method according to claim 14, wherein the separation mixture prior to AEX chromatography is supplemented with an adequate amount of an alkaline solution.

19. Method according to claim 18, wherein the separation mixture prior to AEX chromatography is supplemented with an adequate amount of a solution containing sodium or potassium hydroxide, (or its mono or di basic sodium or potassium salts) or tris(hydroxymethyl)aminomethane

20. A single operational unit which can be used in a method according to claim 13, comprising both an anion exchange chromatography part and a cation exchange chromatography part, which are serially connected, wherein the outlet of the anion exchange chromatography part is connected to the inlet of the cation exchange chromatography part, wherein the unit comprises an inlet at the upstream end of the anion exchange chromatography part and an outlet at the downstream end of the cation exchange chromatography part and wherein the unit also comprises an inlet between the anion exchange chromatography part and the cation exchange chromatography part.

21. A single operational unit which can be used in a method according to claim 14, comprising both an cation exchange chromatography part and a anion exchange chromatography part, which are serially connected, wherein the outlet of the cation exchange chromatography part is connected to the inlet of the anion exchange chromatography part, wherein the unit comprises an inlet at the upstream end of the cation exchange chromatography part and an outlet at the downstream end of the anion exchange chromatography part and wherein the unit also comprises an inlet between the cation exchange chromatography part and the anion exchange chromatography part.
